(19) United States
(12) Patent Application Publication
(10) Pub. No.: US 2005/0181058 A1
(43) Pub. Date: Aug. 18, 2005

(54) Use of polysaccharides, such as galactomannans, glucomannans and the like for introducing active substances into the human or animal metabolism

(76) Inventor: Andreas Hefel, Berlingen (CH)

Correspondence Address: BAKER & DANIELS LLP;111 E. WAYNE STREET, SUITE 800, FORT WAYNE, IN, 46802, US

(21) Appl. No.: 10/780,152

(22) Filed: Feb. 17, 2004

(51) Int. Cl. A61K 9/14

(52) U.S. Cl. 424/488

(57) ABSTRACT

The use of polysaccharides, such as galactomannans, glucomannans and the like, for introducing active substances into the human or animal metabolism is described. The vital substances, individually or as a complex, are embedded separately and in each case functionally separated from one another in a plant-based matrix of the polysaccharide.

U.S. Pat. No. 4,675,312 describes the preparation of polysaccharirde agglomerates.

U.S. Pat. No. 4,675,312 discloses the preparation of an agglomerate with the aim of permitting better consumption by avoiding the other problems of the galactomannan flour, such as viscosity and tack.

The preparation is carried out here by means of two different substances, namely first by means of the galactomannan and secondly by means of agglomeration agents separate therefrom.

The agglomeration agent is scarcely restricted in terms of the choice of the available substances. It is merely defined as a water donor and may be of animal and/or vegetable origin.

The proportion of the agglomeration agent in the granules as a whole is between 5 and 40%. Examples of such agglomeration agents are potatoes, milk and fruits.

U.S. Pat. No. 4,675,312 accordingly describes the preparation of granules from galactomannans and associated agglomeration agents.

However, this publication does not disclose how such granules are used for embedding active substances.

The U.S. patent describes only the use of these granules as roughage. The ready-mix was taken up with liquid which, with the intestinal fluid, helps to swell the product. The health value was therefore limited only to the proportion of roughage forming thereby.

It is the object of the invention further to develop the preparation of polysaccharides, such as galactomannans and glucomannans, described in U.S. Pat. No. 4,675,312, so that they are also suitable for introducing active substances into the human or animal metabolism.

For achieving the object, the invention is characterized by the technical teaching of claim 1.

The use of granules for oral consumption by humans and animals is described. Novel absorption kinetics of water-soluble vital substances is claimed. The delay of the penetration of water into the granules is an advantage with regard to the retarded release of water-soluble vital substances. Fat-soluble vital substances are administered in oily suspension, with the result that the absorption is independent of nutrition.

The invention describes the possibility for the individual composition of the granules described, with their effect on the human organism.

The invention therefore has the following features:
Use of plant ingredients
Polysaccharide carriers
Application in various areas (antiageing, performance sport)

Vital substances are embedded, individually or as a complex, separately in a plant-based matrix (polysaccharides/guar). The advantage is the delayed, retarded release of the vital substances into the blood, the exclusion of undesired interactions of the vital substances with one another (antagonism) and the accumulation in the small intestine.

By producing monopreparations and complexes as semifinished preparations and packing in respective 30 day units, it is possible, in a very simple manner, to prepare completely individual vital substance preparations for individual persons.

The combination of a “modular system” for the simple preparation of individual preparations and the specific embedding of vital substances in plant-based polysaccharides (e.g. guar) is claimed, inter alia, as being essential to the invention.

Introduction of the active ingredient into the polysaccharide: Active substances or vital substances are defined below as substances which may be important for the metabolism. Active substances may be vitamins, minerals, trace elements, plant ingredients, amino acids, coenzymes and other metabolically active substances.

The active substance is dissolved in water or, in the case of fat-soluble active substances, said active substance is suspended in water. This solution or suspension is slowly introduced into the purified polysaccharide and mixed. The resulting gel is dried by a gentle method in order to avoid destroying the active substances, some of which are sensitive, by heat or oxygen.

The cake formed as a result of the drying is comminuted and is sieved to the desired particle size (preferably 0.2-2 mm). The granules thus obtained have a residual moisture content of about 5-7% and are therefore microbiologically stable.

On consumption of the granules, they begin to swell and the embedded active substances are slowly released for absorption by the human or animal digestive system. Owing to the high compaction of the polysaccharide matrix, it is ensured that the swelling process takes place only in the intestinal tract. During the swelling process, water is continuously consumed and the matrix is thus loosened. In the course of this loosening, the embedded active substances can diffuse out of the matrix and hence be absorbed. The amount of active substance which is absorbed therefore does not exceed physiological concentrations, as may occur in the case of the release of active substance from a capsule or conventional dosage form.

The continuous dissolution of the polysaccharide gel by the digestive process results in the delayed release of the embedded active substances. As a result of this behaviour, substantial correspondence to the natural conditions during the consumption of vitamins or other active substances is achieved. Fruit, vegetable, meat and cereal are colloidal systems, as is the hydrocolloid galactomannan or glucomannan.

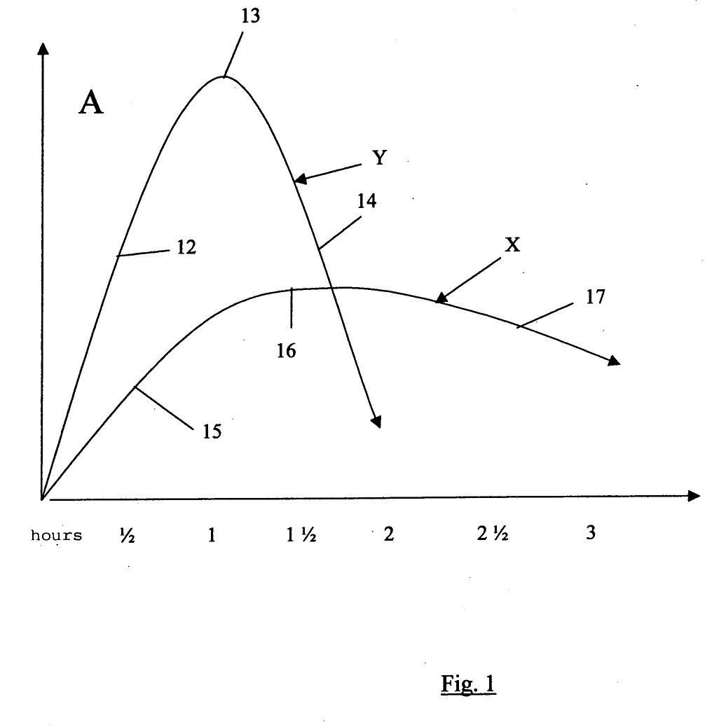

The bioavailability of the embedded active substances is thereby increased. By the consumption of capsules, tablets or powders, practiced to date according to the prior art, the active substance reaches high concentrations in the blood in a time which is physiologically too short and said active substance is therefore also excreted again more rapidly or in some cases is not absorbed at all. A delay in the release of active substance is achieved by the incorporation described. The absorption kinetics achievable by incorporating the active substance into the polysaccharide is shown in FIG. 1.

### EXAMPLE 1

### Preparation of Granules Containing the Active Substance Coenzyme Q10

62 kg of guar flour are initially introduced into a mixer and a solution of 18 kg of coenzyme Q10 and 18 kg of D,L-alpha-tocopherol acetate as an antioxidant in 15 kg of isopropyl alcohol is added. Mixing is carried out and then water is added until the product has reached the maximum moisture content. When water Is added, the polysaccharide matrix begins to swell and the active substance coenzyme Q10 penetrates the polysaccharide chains and is thus immobilized. By subsequent drying under vacuum conditions, the moisture is removed from the product at room temperature to a residual moisture content of 5-7%, and the product is thus stabilized. The cake formed on drying is crushed and is brought to the desired particle size of 0.2 to 2 mm by sieving.

### Example 2

### Preparation of Vitamin C Granules

Dissolution of 10 kg of ascorbic acid in 50 l of water 30 kg of guar flour and 30 kg of konjac flour are initially introduced into a mixer and the ascorbic acid solution is added. During the mixing, the moisture content is if necessary adjusted to the maximum achievable moisture content by addition of water. The mixed material is frozen, comminuted and then dried by lyophilization. The cake formed on drying is crushed and is brought to the desired particle size of 0.2 to 2 mm by sieving.

### Example 3

### Preparation of Trace Element Granules

Preparation of a solution of 480 g of copper sulphate in 10 l of water, of a second solution of 3.2 kg of zinc sulphate heptahydrate in 10 l of water and of a third solution of 5 g of sodium selenite pentahydrate in 5 l of water. 22 kg of guar and 7 kg of potato starch are introduced into a mixer and mixed. The individual solutions are then added in sequence and incorporated. The maximum achievable moisture content is established with water. By subsequent drying in a hot air stream, the moisture is removed from the product to a residual moisture content of 5-7%. The cake formed on drying is crushed and is brought to the desired particle size of 0.2 to 2 mm by sieving.

The following features are therefore claimed as being essential to the invention:
Retardation effect of the incorporated active substances
Prevention of undesired interactions between the active substances, both in the preparation and in the gastrointestinal tract
Natural release behaviour of the carrier (water-soluble, indigestible polysaccharide) and hence improvement of the absorption properties
Improved absorption properties owing to the establishment of a large absorption surface in the small intestine

The subject of the present invention is inherent not only in the subject of the individual Patent Claims but also in the combination of the individual Patent Claims with one another.

All data and features disclosed in the documents, including the Abstract, in particular the three dimensional design shown in the drawings, are claimed as being essential to the invention where, individually or in combination, they are novel compared with the prior art.

The invention is explained in more detail below with reference to drawings representing a plurality of embodiments. Further features and advantages of the invention which are essential to the invention are evident from the drawings and their description.

FIG. 1 shows a comparison of the kinetics of the active substance release in a conventional preparation compared with the active substance on incorporation into a polysaccharide;

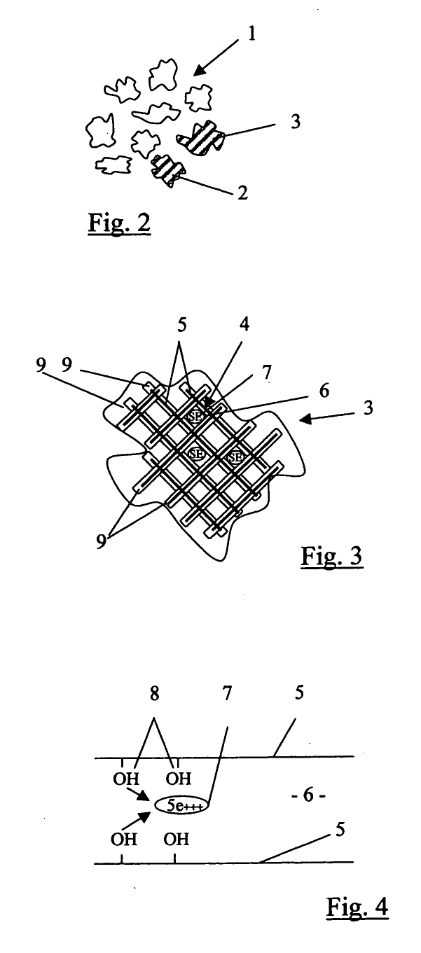

FIG. 2 shows an enlarged, schematic diagram of granules consisting of individual granular particles;

FIG. 3 shows an enlarged and schematic diagram of a granular particle with incorporation of selenite ions;

FIG. 4 shows a schematic diagram even further enlarged compared with FIG. 3;

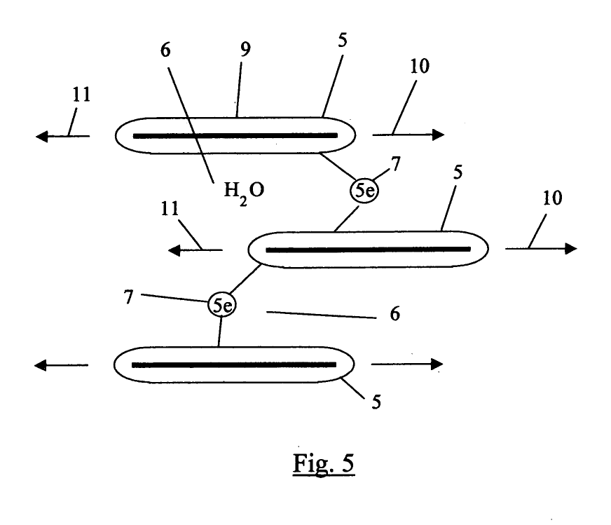

FIG. 5 shows the function kinetics of the molecular synthesis on penetration of water.

FIG. 1 shows a comparison of the release of active substance in the human or animal body via two different active substance mechanisms.

The concentration of active substance in the blood is shown along the ordinate while the time is shown along the abscissa.

The curve Y shows a conventional transfer of an active substance into the human or animal body. From this it is evident that an approximately parabolic curve results, i.e. a very sharp increase of the concentration of active substance to curve branch 12, which culminates in the summit 13 after only one hour and drops off very rapidly in the region of the descending curve branch 14.

From this it is evident that the active substance is available only for a short time.

Furthermore, it is evident from the steep curve branches 12, 14 and the high summit 13 in between that nonphysiologically high active substance concentrations occur—sometimes in ant undesired manner.

The invention is applicable here and, with the flatter curve X, represents an active substance incorporated into a polysaccharide and the transfer of said active substance into the blood of the human or animal body. The concentration of active substance increases over a relatively long time in the region of curve branch 15, there being only a slight summit 16, which proves that there is no risk of undesirably high and nonphysiological overdoses. The decline in active substance in the region of curve branch 17 is also only very slight, so that the diagram in FIG. 1 shows that the relatively high concentration of active substance at the summit 16 is maintained over a very long time.

From the comparison of curve Y with curve X, it is thus evident that, owing to the technical measures according to the invention, a high concentration of active substance in the blood can be achieved over a long period.

The graph clearly shows the possibility of a desired delay of absorption by the embedding of the active substance in a polysaccharide. This means a more uniform supply and better utilization of the active substances in the human and/or animal metabolism.

FIG. 2 shows, as an example, granules 1 which consist of a multiplicity of granular particles 2, 3.

In one granular particle, for example, ascorbic acid is incorporated, as described in the above-mentioned example 2.

In another granular particle 3, for example, a trace element is incorporated, as described below with reference to a selenite ion. This incorporation mechanism is mentioned in Example 3 of the above description.

It is important that the two granular particles 2, 3 are completely functionally separated and do not mix or interact with one another in an undesired manner.

Because the active substances (ascorbic acid and selenite) are bound in different granular particles 2, 3, an undesired interaction between these active substances in the gastrointestinal tract is therefore prevented.

Details of the incorporation of a selenite ion 7 are explained in more detail with reference to FIGS. 3 to 5.

An electron micrograph of a granular particle 3 shows that it is formed from a multiplicity of net-like or lattice-like polysaccharide molecules 5 which form a lattice structure 4.

The selenite ions 7 are bound into the lattice structure 4 of the polysaccharide molecules 5 in the interstices 6 of this lattice structure 4 by a coordinate bond.

It should also be mentioned that the polysaccharide molecules 5 themselves are surrounded by one H₂O envelope each as shown, which envelope completely surrounds and screens the filament-like structure.

In the further enlarged diagram according to FIG. 4, it is evident that 5 OH groups, which are a component of the polysaccharide molecule 5, are attached to the filament-like polysaccharide molecules 5.

The selenite ions 7 are bound in the interstice 6 between the molecules 5 owing to the above-mentioned coordinate bond. Here, the selenite ions are pentavalent and positive while the OH group 8 carries a negative partial charge.

In this way, the selenite ions are held in the interstice 6 between the filament-like polysaccharide ions owing to the coordinate bond described.

The delayed release is thus explained, because the reaction kinetics according to FIG. 5 result on penetration of water into the composite according to FIG. 4.

There, it is once again evident that the polysaccharide molecules 5 surrounded by a water envelope and present in the interstice are bound to one another by molecules of water, in the interstice of which in turn the selenite Ions 7 are also present.

If water or intestinal fluid now penetrates into the interstices 6, partial elimination of the bond between the molecules 5 occurs and these move in two dimensions towards one another in the directions of the arrows 10, 11.

The bond between the polysaccharide molecules 5 is thus partly eliminated, and the selenite ions 7 are released into the surrounding fluid.

This explains the delayed release, because there is still partial adhesion and binding in the interstice 6 between the polysaccharide molecules 5. Furthermore, the delayed release is explained by the fact that the individual filaments are removed layer by layer by the penetrating water or the intestinal fluid, and the lattice structure is thus also removed layer by layer. In order thus to release the selenite ions 7 located in the interstice 6.

The manner in which the hydrate envelope 9 described above occurs is also described below.

In the dry flour, the galactomannan fibers are very closed associated with one another. When water is added to this network, the filaments become loose and are surrounded by the above mentioned hydrate envelope 9.

It is therefore possible in the inventive manner to create the lattice structure of the polysaccharide molecules 5 so that they are surrounded by the hydrate jacket (H₂O envelope 9) mentioned.

The hydrate envelope ensures the intermediate binding between the individual polysaccharide molecules 5, as shown by the reaction kinetics of FIG. 5.

### Drawing Legends

1 Granules
2 Granular particle (Asc)
3 Granular particle (Se)
4 Lattice structure
5 Polysaccharide molecule
6 Interstice
7 Selenite ion
8 OH group
9 H₂O envelope
10 Direction of arrow
11 Direction of arrow
12 Curve branch
13 Summit
14 Curve branch
15 Curve branch
16 Summit
17 Curve branch

1. The use of polysaccharides, such as galactomannans, glucomannans and the like, for introducing active substances into the human or animal metabolism, characterized in that the vital substances, individually or as a complex, are embedded separately and in each case functionally separated from one another in a plant-based matrix of the polysaccharide.

2. Use according to claim 1, characterized in that the vital substances are vitamins, minerals, trace elements, plant ingredients, amino acids, coenzymes and other metabolically active substances.

3. Use according to claim 1, characterized in that
the active substance is dissolved in water or, in the case of fat-soluble active substances, it is suspended in water,
the solution or suspension is introduced slowly into the purified polysaccharide and mixed,
the resulting gel is dried by a gentle method,
the cake formed as a result of the drying is comminuted and
is sieved to the desired particle, size (preferably 0.2-2 mm).

4. Polysaccharide according to claim 1, characterized in that granules consist of a multiplicity of granular particles, a first active substance is incorporated in a first granular particle and a second active substance is incorporated in a second granular particle.

5. Polysaccharide according to claim 4, characterized in that the granular particles are functionally separated and do not mix or interact with one another in an undesired manner.

6. Polysaccharide according to claim 1, characterized in that the granular particles are formed from a multiplicity of net-like or lattice-like polysaccharide molecules which form a lattice structure and that the active substance ions are bound into the lattice structure of the polysaccharide molecules in the interstices of the lattice structure by a coordinate bond.

7. Polysaccharide according to claim 1, characterized in that the polysaccharide molecules are surrounded by an H₂O envelope which completely surrounds and screens the filament-like structure.

8. Polysaccharide according to claim 1, characterized in that OH groups arc attached to the filament-like polysaccharide molecules, and that the active substance ions are bound in the interstice by a coordinate bond.

9. Polysaccharide according to claim 1, characterized in that, on penetration of water or intestinal fluid into the interstices of the molecules, the latter move in two dimensions towards one another (in the directions of arrows 10, 11).

10. Polysaccharide according to claim 1, characterized in that the delayed release of the active substances takes place as a result of the fact that the individual filaments are removed layer by layer by the penetrating water or the intestinal fluid, and the lattice structure is thus also removed layer by layer in order thus to release the active substance ions located in the interstice.

11. Polysaccharide according to claim 1, characterized in that the filament-like molecules are surrounded by a hydrate jacket (H₂O envelope 9).

12. Use according to claim 2, characterized in that
the active substance is dissolved in water or, in the case of fat-soluble active substances, it is suspended in water,
the solution or suspension is introduced slowly into the purified polysaccharide and mixed,
the resulting gel is dried by a gentle method,
the cake formed as a result of the drying is comminuted and
is sieved to the desired particle, size (preferably 0.2-2 mm).

13. Polysaccharide according to claim 2, characterized in that granules consist of a multiplicity of granular particles, a first active substance is incorporated in a first granular particle and a second active substance is incorporated in a second granular particle.

14. Polysaccharide according to claim 3, characterized in that granules consist of a multiplicity of granular particles, a first active substance is incorporated in a first granular particle and a second active substance is incorporated in a second granular particle.

15. Polysaccharide according to claim 2, characterized in that the granular particles are formed from a multiplicity of net-like or lattice-like polysaccharide molecules which form a lattice structure and that the active substance ions are bound into the lattice structure of the polysaccharide molecules in the interstices of the lattice structure by a coordinate bond.

16. Polysaccharide according to claim 3, characterized in that the granular particles are formed from a multiplicity of net-like or lattice-like polysaccharide molecules which form a lattice structure and that the active substance ions are bound into the lattice structure of the polysaccharide molecules in the interstices of the lattice structure by a coordinate bond.

17. Polysaccharide according to claim 4, characterized in that the granular particles are formed from a multiplicity of net-like or lattice-like polysaccharide molecules which form a lattice structure and that the active substance ions are bound into the lattice structure of the polysaccharide molecules in the interstices of the lattice structure by a coordinate bond.

18. Polysaccharide according to claim 5, characterized in that the granular particles are formed from a multiplicity of net-like or lattice-like polysaccharide molecules which form a lattice structure and that the active substance ions are bound into the lattice structure of the polysaccharide molecules in the interstices of the lattice structure by a coordinate bond.

19. Polysaccharide according to claim 2, characterized in that the polysaccharide molecules are surrounded by an H₂O envelope which completely surrounds and screens the filament-like structure.

20. Polysaccharide according to claim 3, characterized in that the polysaccharide molecules are surrounded by an H₂O envelope which completely surrounds and screens the filament-like structure.
